(19) United States
(12) Patent Application Publication
(10) Pub. No.: US 2011/0281946 A1
(43) Pub. Date: Nov. 17, 2011

(54) POLYMERIC -BASED COMPOSITION

(75) Inventor: Alan John WADDON, Derbyshire (GB)

(73) Assignee: NORTHERN HYDROGELS LTD, Sheffield (GB)

(21) Appl. No.: 13/105,229

(22) Filed: May 11, 2011

(30) Foreign Application Priority Data
May 12, 2010 (GB) 1007929.1

(51) Int. Cl. A61K 31/23; A61K 31/07; A61K 31/167; A61P 27/02; A61Q 19/00; A61Q 19/08; A61P 31/00; A61P 23/00; A61K 31/192; A61K 8/90

(52) U.S. Cl. 514/552; 514/570; 514/725; 514/626

(57) ABSTRACT

The present invention concerns polymeric-based compositions that are useful in the delivery and sustained release of hydrophobic therapeutic and/or cosmetic agents substantially at the point of injury, infection or, in the case of purely cosmetic applications, the point of treatment. Also provided are methods of preparing such compositions and methods of treatments involving the use of such compositions.

The present invention concerns polymeric based compositions that are useful in the delivery and sustained release of therapeutic and/or cosmetic agents substantially at the point of injury, infection or, in the case of purely cosmetic applications, the point of treatment. Also provided are methods of preparing such compositions and treatments involving the use of such compositions.

Water soluble polymers are used in the food, cosmetic, dermal filler, personal care, textile manufacture industries as well as the wound care, drug release device and contact lens industries. In some cases water soluble polymers can form polymeric hydrogels.

Polymeric hydrogels are three dimensional (synthetic or natural) networks which are capable of absorbing or releasing large amounts of water. Polymeric hydrogels are used in the food, cosmetic, dermal filler, personal care, textile manufacture industries as well as the wound care, drug release device and contact lens industries.

In drug delivery applications, hydrogels loaded with therapeutic reagents are applied locally (e.g. Hubbell, J. A. (1996) J. Contr. Rel,. 39, 305-313). Application may be oral, topical, rectal, vaginal or otherwise in the body. In non-oral administration, therapeutic reagents are released from the hydrogel directly to the target site.

Solutions based on the amphiphilic triblock copolymers Pluronic® F68 or Pluronics® F127, available from BASF, are known to be potentially useful to prepare topical drug delivery systems such as for ibuprofen and sodium ibuprofen. Solutions based on the amphiphilic triblock copolymers Pluronic® F68 or Pluronics® F127 may also form thermo-responsive aqueous hydrogels. In such systems, it is believed that the amphiphilic triblock copolymer forms micelles in the water providing a hydrogel. The hydrogel comprises a plurality of hydrophobic inner cores surrounded by hyrodrophilic outer corona. It is believed that ibuprofen, which is extremely hydrophobic and insoluble in water, becomes dissolved in the hydrophobic core of the micelles to form ‘nano-packets’, whereas the polar sodium ibuprofen is believed to be dissolved in the hydrophilic aqueous matrix. (A hydrogel drug delivery system for topical application of Ibuprofen, Waddon and MacNeil, University of Sheffield, UK, poster at Polymer Showcase, Wakefield, UK, 2005)

U.S. Pat. No. 4,690,823 discloses an ibuprofen-containing soft gelatin capsule for oral administration containing a solution of from 15-30 parts by weight ibuprofen in from 70-85 parts by weight of polyoxyethylene-polyproplene polymer (e.g. Pluronic® L42, available from BASF) or 30-76 parts by weight of polyalkelyne gycol and 7-40 parts by weight of a surfactant, e.g. Cremophor® RH40, available from BASF) for the treatment of rheumatic diseases.

Hyaluronic acid (HA) is a molecule composed of 250 to 25,000 β-1,4 linked disaccharide units consisting of D-glucuronic acid and N-acetyl-D-glucosamine linked by a β-1,3 glycosidic bond. It is known that at low shear rates, HA molecules in aqueous solutions form tangled masses that greatly impede flow resulting in viscous solutions. This viscoelastic behaviour is disclosed to make hyaluronate solutions excellent biological shock absorbers and lubricants. It is also known that HA is commonly found in synovial fluid and skin. (Biochemistry, 2^{nd} Ed., Voet and Voet, John Wiley & Sons, Inc., 1995, pp 264-265).

HA also has properties of hydrating bodily tissue and is used in cosmetic creams and lotions to hydrate skin and lips and improve visual appearance. HA is known for its beneficial effect of reducing the appearance of wrinkles of the skin. It is believed this occurs by stimulating production of collagen. HA is used in topical anti aging creams. HA is also used in ophthalmic solutions.

Retinol is the animal form of vitamin A. It is a fat-soluble vitamin important in vision and bone growth. It has a molecular weight of 286. Retinol is insoluble in water.

Retinol belongs to the class of compounds called retinoids which also include retinal (retinaldehyde), retinoic acid and retinyl esters (e.g. retinyl palmitate and retinyl acetate). All retinods are forms of Vitamin A.

Retinoids administered in the form of topical creams are known to reduce the appearance of wrinkles. Retinol, retinyl palmitate and retinoic acid are commercially available in topically applied creams. The most preferred form of retinoid for anti-aging is retinol in creams of concentrations of less than 2%.

It is desirable to incorporate dissolved HA and dissolved retinol or retinyl palmitate in the same topical composition. It is preferable that the topical preparation be water based. It is also desirable to incorporate both dissolved HA and dissolved retinol or retinyl palmitate in a composition that is injected into the dermal layer. Preferably, the injectable composition is water based. However, retinol and retinyl palmitate are insoluble water.

US20090018102 (application) discloses a dermatologically effective amount of hyaluronic acid, at least one retinoid and/or salt and/or derivative, at least one oligosaccharide and at least one inhibitor of hyaluronic acid degradation. However, no means of dissolving hydrophobic materials in the aqueous medium is disclosed.

U.S. Pat. No. 5,095,037 discloses a pharmaceutical composition for treating inflammatory diseases, comprising HA or its salt in combination with an anti-inflammatory agent.

WO-A-06039704 discloses unencapsulated and encapsulated therapeutic agents, e.g. ibuprofen, in admixture with an HA delivery vehicle. The particles (e.g. 0.5-200 μm) of unencapsulated agent or encapsulated therapeutic agents, e.g. suprofen, may be suspended in a natural or synthetic biodegradable polymer, e.g. PCL-GA-LA polymer (MW 12400, 30% by weight) in benzyl benzoate (65% by weight), and then dissolved or suspended in the HA delivery vehicle. The composition used to disperse the therapeutic agents is not a hydrogel because it contains no water, water only being present in the final HA delivery vehicle. The composition is for treating joint capsule arthropy.

US 2005/0164980-A1 discloses pharmaceutical preparations comprising HA modification products, in which HA and/or a pharmaceutically acceptable salt thereof is chemically bonded to a block copolymer such as Pluronic® ethylene oxide-propylene oxide-ethylene oxide block copolymers. Such modification products, when injected into a living body, have a residence time exceeding that of conventional HA preparations, for example a period of two weeks or more. It is also disclosed that such HA modification products undergo phase transition at body temperature, sharply increasing its viscoelasticity, i.e. the HA modification product is not in a state of gelation below its phase transition temperature but in a liquid-like sol state with some fluidity. It is suggested that the HA modification products could be applicable to a carrier for sustained released drugs.

There is a need for a new composition, which is preferably solvent-free, for the targeted delivery and/or sustained release of therapeutic and/or cosmetic agents, especially hydrophobic therapeutic and/or cosmetic agents, substantially to the point of infection, injury or, in the case of purely cosmetic applications, the point of treatment which are improved in performance over the prior art compositions and/or simpler to manufacture.

The present invention, in its various aspects, is as set out in the accompanying claims.

In a first aspect, the present invention provides an aqueous composition consisting of:
i) water
ii) at least one amphiphilic block copolymer dissolved in i) capable of solubilising iv) when dissolved in i);
iii) hyaluronic acid (HA) or a pharmaceutically acceptable salt thereof dissolved in i);
iv) at least one hydrophobic therapeutic and/or cosmetic agent solubilised in ii) and optionally
v) at least one other ingredient that is not i), ii), iii), iv) or an organic solvent or other solubilising agent or carrier for said hydrophobic therapeutic and/or cosmetic agent;
wherein there are no chemical bonds between ii) and iii).

Preferably the at least one amphiphilic block copolymer comprises a hydrophilic component selected from poly ethylene oxide (PEO), poly vinyl alcohol (PVA), poly glycolic acid (PGA), poly (N-isopropylacrylamide), poly(acrylic acid) (PAA), poly vinyl pyrrolidone (PVP) or mixtures thereof. More preferably, the amphiphilic block copolymer comprises a hydrophilic component comprising polyethylene oxide (PEO).

Preferably the at least one amphiphilic block copolymer comprises a hydrophobic component selected from polypropylene oxide (PPO), poly (lactic acid) (PLA), poly (lactic acid co glycolic acid) (PLGA), poly (β-benzoyl L-aspartate) (PBLA), poly (γ-benzyl-L-glutamate) (PBLG), poly (aspartic acid), poly (L lysine), poly(spermine), poly (caprolactone) and mixtures thereof. More preferably, the amphiphilic block copolymer comprises a hydrophobic component comprising polypropylene oxide (PPO).

Preferably, the at least one amphiphilic block copolymer comprises a hydrophilic component selected from poly ethylene oxide (PEO), poly vinyl alcohol (PVA), poly glycolic acid (PGA), poly (N-isopropylacrylamide), poly(acrylic acid) (PAA), poly vinyl pyrrolidone (PVP) or mixtures thereof, and a hydrophobic component selected from polypropylene oxide (PPO), poly (lactic acid) (PLA), poly (lactic acid co glycolic acid) (PLGA), poly (β-benzoyl L-aspartate) (PBLA), poly (γ-benzyl-L-glutamate) (PBLG), poly (aspartic acid), poly (L-lysine), poly(spermine), poly (caprolactone) or mixtures thereof. Examples of such amphiphilic block copolymers include (PEO)(PPO)(PEO) block copolymers (PEO/PPO), and poly (lactic acid co glycolic acid) block copolymers (PLGA), such as (PEO)(PLGA)(PEO) block copolymers.

More preferably the at least one amphiphilic block copolymer is a compound of Formula 1, 1′, 2 or 2′ below, or a mixture thereof:

HO[CH₂—CH₂O]ₘ[CH(CH₃)—CH₂O]ₙ[CH₂CH₂O]_{m′}H Formula 1

HO[CH(CH₃)—CH₂O]ₙ[CH₂CH₂O]ₘ[CH(CH₃)—CH₂O]_{n′}H Formula 1′

wherein m and m′ independently represent an integer from about 50 to about 125, e.g. from about 60 to about 115, and n independently represents an integer from 10 to about 80, e.g. from about 20 to about 75;

HO[CH₂—CH₂—O]ᵣ—{[CHCH₃—CO—O]_{q}—[CH₂—CO—O]ₚ}—[CH₂—CH₂O]_{r′}H Formula 2

HO[CO—CH(CH₃)—O]_{q}—[CO—CH₂—O]ₚ—[CH₂—CH₂—O]ᵣ—[CO—CH(CH₃)—O]_{q′}—[CO—CH₂—O]_{p′}—H Formula 2′

wherein p and p′ independently represent an integer from about 5 to about 50, q and q′ independently represent an integer from about 5 to about 50, and r and r′ independently represent an integer from about 10 to about 125.

The most preferred amphiphilic block copolymers are of Formula 1 and Formula 2. Examples of copolymers of Formula 1 are BASF's Pluronic® F127, which is believed to have the formula where m and m′ are in the range 85-110 and n is in the range 50-70, and Pluronic F68, which is believed to have the formula wherein m and m′ are in the range 65-85 and n is in the range 20-40.

The hyaluronic acid (HA) or a pharmaceutically acceptable salt thereof may be present in the pharmaceutical or cosmetic composition in either an uncrosslinked or a crosslinked state, or as a mixture of these states.

The compositions of the present invention enable site-specific delivery and/or sustained release of one or more hydrophobic therapeutic and/or cosmetic agents, optionally present in a mixture with one or more hydrophilic therapeutic and/or cosmetic agents. The skilled person will readily understand what is meant by the terms hydrophobic and hydrophilic, but for the avoidance of doubt, the term hydrophobic refers to any compound which is insoluble or poorly soluble, e.g. less than 0.1 gL⁻¹, in water.

The compositions of the present invention enable the solubilisation of hydrophobic therapeutic and/or cosmetic agents to a solute concentration of greater than 0.1 gL⁻¹ without the use of a non-aqueous solvent or other solubilising agent or carrier for such hydrophobic agents. In this regard, the skilled person will readily understand what is meant by the term ‘solubilised’, but for the avoidance of doubt, and although gentle heating, such as up to and including 80° C., may be required to form a solution comprising the hydrophobic therapeutic and/or cosmetic agent in the amphiphilic block copolymer and water, the term solubilised refers to a hydrophobic therapeutic and/or cosmetic agent which remains in the resultant solution for a period of at least 12 months at a temperature of 10° C.

Therapeutic and/or cosmetic agents typically suitable for use in the present invention have a low molecular mass, i.e. less than 1000. Suitable hydrophobic therapeutic agents include, but are not limited to compounds such as lidocaine or ibuprofen. Suitable cosmetic agents, either hydrophobic or hydrophilic, include, but are not limited to anti-aging agents selected from the group consisting of retinol, retinal, retinyl palmitate, retinyl acetate, other esters of retinol, retinoic acid, other retinoids, collagen, vitamin E, vitamin C, other vitamins, idebenone, peptides, pentapeptides, polypeptides, transcription factors, UV blockers and mixtures thereof.

In one embodiment, a mixture of hydrophobic and hydrophilic therapeutic and/or cosmetic agents are present in the composition of the present invention. Suitable hydrophilic therapeutic agents include, but are not limited to, pharmaceutically acceptable salts of lidocaine or ibuprofen, e.g. sodium ibuprofen.

Any therapeutic agents, either hydrophobic or hydrophilic, are preferably selected from the group consisting of analgesics, antimicrobial agents, hormones, enzymes, antiallergic agents, antibiotics, steroidal anti-inflammatory agents, non-steroidal anti-inflammatory agents, cytokines, growth factors, antineoplastic agents, anaesthetics, astringents, immunosuppressants, anticoagulants, anticoagulation factors, clotting factors, anti-oxidants and mixtures thereof.

In one embodiment, at least one hydrophobic therapeutic agent is present in the composition of the present invention. In one such embodiment, the at least one hydrophobic therapeutic agent is a non-steroidal anti-inflammatory agent, such as ibuprofen. In another such embodiment, the at least one hydrophobic therapeutic agent is an anaesthetic, such as lidocaine.

In one embodiment, at least one hydrophobic cosmetic agent is present in the composition of the present invention. In one such embodiment, the at least one hydrophobic cosmetic agent is retinol. In another such embodiment, the at least one hydrophobic cosmetic agent is retinyl palmitate.

In one embodiment, the composition of the present invention comprises at least one therapeutic agent that is a polypeptide, protein, DNA or RNA, or a mixture thereof.

In another embodiment, the composition comprises at least two therapeutic and/or cosmetic agents, provided that one or more solubilised therapeutic and/or cosmetic agent present in the composition is hydrophobic. For example, the composition may comprise a non-steroidal anti-inflammatory agent, such as ibuprofen, and an anaesthetic, such as lidocaine, and/or their pharmaceutically acceptable salts. As a further example, the composition may comprise an anaesthetic, such as lidocaine, and a cosmetic agent, such as retinol.

In another embodiment, the composition of the present invention is a dermal filler, which may be used to reduce or reduce the appearance of wrinkles on facial or other areas of the body.

The compositions of the present invention are free of organic solvent or other solubilising agent or carrier for the hydrophobic therapeutic and/or cosmetic agent. In this regard, it will be appreciated that whilst no physiologically incompatible organic solvent or other organic solubilising agent, such as ethanol, propanol, ethylene glycol, methylene chloride and benzyl benzoate, is intended to be used in the formulation of the compositions, residual amounts of such organic solvents and solubilising agents may be present depending upon the purity of the components employed in the formulation of the composition. Such solvent-free compositions are beneficial as they are readily suitable for in vivo medical applications, such as intra-articular injections or as dermal fillers, and for topical medical applications, such as for the treatment of wounds where the epidermis has been ruptured (a patient can suffer painful stinging sensations when open wounds are exposed to organic solvents).

In another embodiment, the composition of the present invention is capable of forming a polymeric hydrogel at body temperature.

Preferably, such hydrogel-forming compositions of the present invention are thermoresponsive. By this we mean that the composition is not in a state of gel at temperatures below its phase transition temperature but in a state of sol with fluidity, and at temperatures above its phase transition temperature it is in the form of a hydrogel. As a person skilled in the art will fully appreciate, the phase transition temperature of the thermoresponsive composition should be below body temperature. A thermoresponsive composition according to the present invention preferably has a viscosity at 5° C. of no more than 1000 mPa·s and at 37.5° C. of no less than 10,000 mPa·s.

By body temperature, we mean those temperatures normally associated with the core temperature of the human body. Typically, body temperature will be in the range 35-40° C., with about 37.5° C. being considered normal.

The present composition is surprisingly beneficial over comparable prior art equivalents, in that the present composition can provide for the solubization of hydrophobic therapeutic and/or cosmetic agents in an aqueous medium without the use of a physiologically incompatible organic solvent or other organic solubilising agent. Thus the present invention provides a solution to a long standing and well recognised clinical problem which the prior art had not previously addressed.

The thermoresponsive hydrogel-forming embodiments of the present invention further provide additional benefits because, as injectable compositions, they can be readily prepared and injected at temperatures lower than body temperature yet form gels when in the body which assist sustained release of the therapeutic and/or cosmetic agents and resist dispersion of the compositions into the body, thereby retaining the composition at the locus of treatment. Compositions of the present invention may be used in vivo, for example for the treatment of arthritic pain or the treatment of internal pain caused by surgery on a joint or other internal structure or organ, or they may be used as dermal fillers, for example to remove wrinkles in the skin, or they may be used to treat infections or injury in mucosal passageways. The compositions may also be used topically, for example in ophthalmic compositions, cosmetic compositions and in the treatment of skin wounds, especially where the epidermis has been ruptured.

In a second aspect, the present invention provides a process for preparing a composition according to the first aspect of the present invention, comprising mixing said water, said at least one amphiphilic block copolymer, said at least one hydrophobic therapeutic and/or cosmetic agent and, if present, any optional ingredients that are not an organic solvent or other solubilising agent or carrier for the hydrophobic therapeutic and/or cosmetic to form an aqueous solution, suspension or dispersion, and mixing and dissolving the hyaluronic acid or a pharmaceutically acceptable salt thereof in said aqueous solution, suspension or dispersion.

The process for preparing a thermoresponsive composition preferably comprises dissolving the amphiphilic block copolymer at a temperature wherein the viscosity of the resultant solution is less than 1000 mPa·s. In one embodiment of this process, iii), iv) and, if any, v) are added to a solution of amphiphilic block copolymer in water at a temperature wherein the viscosity of the final solution, suspension or dispersion is less than 1000 mPa·s. In another embodiment of this process, iii), iv) and, if any, v) is/are dissolved, suspended and/or dispersed in i) prior to addition of amphiphilic block copolymer, said addition of the amphiphilic block copolymer being effected at a temperature wherein the viscosity of the final solution, suspension or dispersion is less than 1000 mPa·s.

The process of the present invention is particularly beneficial as it can avoid the need to use potentially hazardous and expensive organic solvents.

In a third aspect, the present invention provides a method of treating a patient suffering from or potentially suffering from internal injury or infection comprising administering a composition according to the first aspect of the present invention into the body of the patient substantially at the point of injury or infection or near there to.

The methods of treatment of the present invention may be suitable for either prophylactic treatment and/or for administration following localised infection or injury. The patient for treatment is generally a mammal, usually a human but other mammals may also be treated, e.g. a horse. The methods are particularly suitable for the treatment of injury or infection to synovial joints such as those found in the (fingers, thumbs, hand, wrist, elbow, shoulder, toes, foot, ankle, knee, hip). Preferably, the compositions of the present invention are administered via intra-articular injection such that the compositions enter and are retained within the articular cavity of the target joint.

Preferably, the methods of the present invention comprise administering a thermoresponsive composition according to the first aspect of the present invention. In such an embodiment, it is preferable that the composition is administered when the composition is at a temperature where the composition is not in a hydrogel state, for example the composition may be at room temperature or lower (e.g. 5-10° C.) depending upon the formulation of the composition. After administration, as the composition warms towards body temperature, the viscosity of the composition will increase and form a hydrogel, retaining the composition within the articular cavity of the target joint.

In a fourth aspect, the present invention provides a method of reducing wrinkles or the appearance of wrinkles on facial or other areas of the body comprising injecting a composition according to the first aspect of the present invention into the skin below the surface of the skin of a patient at a point substantially below said wrinkle or near there to.

In a fifth aspect, the present invention provides a method of reducing wrinkles or the appearance of wrinkles on facial or other areas of the body comprising the topical application of a composition according to the first aspect of the present invention onto said facial or other area of the body.

In a sixth aspect, the present invention provides a method of treating a patient suffering from or potentially suffering from at least one ailment of the eye comprising administering a composition according to the first aspect of the present invention into the interior or onto the surface of the eye. In this aspect, the composition can be applied topically or by injection. Preferably, the composition is applied topically.

In each aspect of the present invention, it should be understood that there are no direct or indirect chemical bonds (i.e. covalent bonds) existing between HA and the amphiphilic block copolymer.

The present invention shall now be more specifically described and explained by way of the following non-limiting examples:

### EXAMPLE 1

### Preparation of 2% Ibuprofen Composition

### a) Dissolution of Pluronics F127 in Water

Pluronics F127 powder (from Sigma-Aldrich) is added with gentle stirring to distilled water at 5° C. to form a 30% (by weight) mix. This achieved, for example, by adding 30 g of Pluronics F127 powder to 70 g of distilled water. The mix is held at 5° C. with occasional gentle stirring for 3 days until it forms a clear, flowable solution.

### b) Dissolution of Ibuprofen in Pluronics F127 Solution

0.497 g of finely ground ibuprofen powder (from Sigma-Aldrich) is added to 24.939 g of 30% Pluronics F127 solution at 5° C. The mixture is shaken to disperse the ibuprofen in the liquid. The sample is stirred at 80° C. for 1-3 hours until the ibuprofen fully dissolves in a solid gel. Air bubbles introduced by the stirring are removed by cooling the gel to 5° C. where it changes to a liquid state where the air bubbles are able to escape.

### c) Addition of Hyaluronic Acid

0.349 g of dry, uncrosslinked hyaluronic acid in solid, powder form (from Sigma-Aldrich) is added to the solution of ibuprofen and Pluronics F127 in water. This is done at ˜5° C. The quantity of HA added is based on the quantity of water present and is calculated to be equal to 2% of the weight of the water.

The material is held for ˜1 day at 5° C.; it is then stirred at ˜70° C. for ˜30 minutes to form a clear transparent material. This is stored at room temperature.

The composition of the final formulation in Example 1 is:

| | |
|---|---|
| Water | 17.457 g |
| Pluronics F127 | 7.482 g |
| Ibuprofen | 0.497 g |
| Hyaluronic Acid | 0.349 g |

### EXAMPLE 2

### Preparation of 1.5% Ibuprofen Composition

### a) Preparation of 25% Pluronics Solution

1.546 g of distilled water is added to 7.850 g of 30% Pluronics solution at 5° C. (see Example 1). This makes a solution in which the final concentration of Pluronics F127 is 25% (by weight) and which has a weight of 9.396 g

### b) Dissolution of Ibuprofen in Pluronics F127 Solution

0.139 g of finely ground ibuprofen powder (Sigma-Aldrich) is added to 9.396 g of the 25% Pluronics F127 solution at 5° C. The mixture is mixed to disperse the ibuprofen in the liquid. The sample is stirred at 80° C. for ˜1-3 hours until the ibuprofen fully dissolves in a solid gel. Air bubbles introduced by the stirring are removed by cooling the gel to a liquid state (at 5° C.) where the air bubbles are able to escape.

### c) Addition of Hyaluronic Acid

0.137 g of uncross-linked hyaluronic acid (Sigma-Aldrich) is added to the solution of ibuprofen, Pluronics F127 in water held at 5° C. The material is held for ˜1 day at 5° C.; it is then stirred at ˜70° C. for ˜30 minutes to form a clear transparent material. This is stored at room temperature.

The composition of the final formulation in Example 2 is:

| | |
|---|---|
| Water | 1.546 g |
| 30% Pluronics F127 solution | 7.850 g |
| Ibuprofen | 0.139 g |
| Hyaluronic Acid | 0.137 g |

### EXAMPLE 3

### Preparation of 1.0% Ibuprofen Composition

### a) Preparation of 20% Pluronics Solution

4.990 g of distilled water is added to 9.996 g of 30% Pluronics solution at 5° C. (see Example 1). This makes a solution in which the final concentration of Pluronics F127 is 20% (by weight) and which has a weight of 14.986 g

### b) Dissolution of Ibuprofen in Pluronics F127 Solution

0.150 g of finely ground ibuprofen powder (Sigma-Aldrich) is added to 14.986 g of the 20% Pluronics F127 solution at 5° C. The mixture is mixed to disperse the ibuprofen in the liquid. The sample is mixed to 80° C. for 1-3 hours until the ibuprofen fully dissolves in a solid gel. Air bubbles introduced by the stirring are removed by cooling the gel to a liquid state (at 5° C.) where the air bubbles are able to escape.

### c) Addition of Hyaluronic Acid

0.240 g of uncross-linked hyaluronic acid (Sigma-Aldrich) is added to the solution of ibuprofen, Pluronics F127 in water at 5° C. The material is held for ˜1 day at 5° C.; it is then stirred at ˜80° C. for ˜30 minutes to form a clear transparent material. This is stored at room temperature.

The composition of the final formulation in Example 3 is:

| | |
|---|---|
| Water | 4.990 g |
| 30% Pluronics F127 solution | 9.996 g |
| Ibuprofen | 0.150 g |
| Hyaluronic Acid | 0.240 g |

### EXAMPLE 4

### Preparation of 0.5% Ibuprofen Composition

### a) Preparation of 10% Pluronics Solution

7.426 g of distilled water is added to 7.527 g of 20% Pluronics solution at 5° C. (see Example 3). This makes a solution in which the final concentration of Pluronics F127 is 10% (by weight) and which has a weight of 14.953 g

### b) Dissolution of Ibuprofen in Pluronics F127 Solution

0.073 g of finely ground ibuprofen powder (Sigma-Aldrich) is added to 14.953 g of the 10% Pluronics F127 solution at 5° C. The mixture is mixed to disperse the ibuprofen in the liquid. The sample is mixed at 80° C. for 45 minutes until the ibuprofen fully dissolves. Air bubbles introduced by the stirring are removed by cooling the gel to 5° C.) where the air bubbles are able to escape.

### c) Addition of Hyaluronic Acid

0.2660 g of uncross-linked hyaluronic acid (Sigma-Aldrich) is added to the solution of ibuprofen, Pluronics F127 in water held at 5° C. The material is held for ˜1 day at 5° C.; it is then stirred at ˜80° C. for ˜30 minutes to form a clear transparent material. This is stored at room temperature.

The composition of the final formulation in Example 4 is:

| | |
|---|---|
| Water | 7.426 g |
| 20% Pluronics F127 solution | 7.527 g |
| Ibuprofen | 0.073 g |
| Hyaluronic Acid | 0.266 g |

### EXAMPLE 5

### Preparation of Dermal Compositions Containing Retinyl Palmitate

### a) Preparation of Pluronics F127 Solution

Distilled water is added to Pluronics F127 powder at 5° C. (see Example 3) to make solutions in which the final concentration of Pluronics F127 is either 10% or 15% (by weight).

### b) Dissolution of Retinyl Palmitate/Tocepherol in Pluronics F127 Solution

Tocopherol stabilised retinyl palmitate (supplied by Adina Chemicals) is added to either 10% or 15% of Pluronics F127 solution at 5° C. The sample is mixed at 80° C. for 30 minutes with occasional ultrasonic mixing until the retinyl palmitate fully dissolves.

The solutions are then cooled to 5° C.

### c) Addition of Hyaluronic Acid

Uncross-linked hyaluronic acid (Falcon Trading International, VT, USA) is added to the solution of tocopherol stabilised retinyl palmitate and Pluronics F127 in water held at 5° C. The material are held for ˜2 days at 5° C. to form a solution.

The compositions of the final formulation in Example 5 is:

(a)

| | |
|---|---|
| 10% Pluronics F127 solution | 19.993 g |
| Tocopherol stabilised retinyl palmitate | 0.055 g |
| Hyaluronic Acid | 0.101 g |

(b)

| | |
|---|---|
| 15% Pluronics F127 solution | 19.980 g |
| Tocopherol stabilised retinyl palmitate | 0.115 g |
| Hyaluronic Acid | 0.101 g |

### EXAMPLE 6

### Preparation of Dermal Composition Containing Retinol

### a) Preparation of Pluronics F127 Solution

Distilled water is added to Pluronics F127 powder at 5° C. (see Example 3) to make solutions in which the final concentration of Pluronics F127 is 10% (by weight).

### b) Dissolution of Retinol in Pluronics F127 Solution

Retinol (supplied by Sigma-Aldrich) is added to 10% Pluronics F127 solution at 5° C. The sample is mixed at 80° C. until the retinol fully dissolves (1-2 hours). The solutions are then cooled to 5° C.

### c) Addition of Hyaluronic Acid

Uncross-linked hyaluronic acid (Falcon Trading International, VT, USA) is added to the solution of retinol and Pluronics F127 in water held at 5° C. The material is held for ˜2 days at 5° C. to form a solution.

The composition of the final formulation in Example 6 is:

| | |
|---|---|
| 10% Pluronics F127 solution | 10.047 g |
| Tocopherol stabilised retinyl palmitate | 0.050 g |
| Hyaluronic Acid | 0.052 g |

1. An aqueous composition consisting of:
i) water
ii) at least one amphiphilic block copolymer dissolved in i) that is capable of solubilising iv) when dissolved in i);
iii) hyaluronic acid (HA) or a pharmaceutically acceptable salt thereof dissolved in i);
iv) at least one hydrophobic therapeutic and/or cosmetic agent solubilised in ii); and optionally
v) at least one other ingredient that is not i), ii), iii), iv) or a solvent or other solubilising agent or carrier for said hydrophobic therapeutic agent;
wherein there are no chemical bonds between ii) and iii).

2. A composition according to claim 1, wherein said amphiphilic block copolymer comprises a hydrophilic component comprising polyethylene oxide) (PEO), poly vinyl alcohol (PVA), poly (glycolic acid) (PGA), poly (N-isopropylacrylamide), poly(acrylic acid) (PAA), poly vinyl pyrrolidone (PVP) or mixtures thereof.

3. A composition according to claim 2, wherein said amphiphilic block copolymer comprises a hydrophilic component comprising polyethylene oxide (PEO).

4. A composition according to claim 1, wherein said amphiphilic block copolymer comprises a hydrophobic component comprising polypropylene oxide (PPO), poly (lactic acid) (PLA), poly (lactic co glycolic) acid (PLGA), poly (β-benzyl L-aspartate) (PBLA), poly (γ-benzyl-L-glutamate) (PBLG), poly (aspartic acid), poly (L-lysine), poly (spermine), poly (caprolactone) and mixtures thereof.

5. A composition according to claim 4, wherein said amphiphilic block copolymer comprises a hydrophobic component comprising polypropylene oxide (PPO), poly (lactic co glycolic) acid (PLGA) or mixtures thereof.

6. A composition according to claim 1, wherein said amphiphilic block copolymer is a compound of Formula 1 or 1′, or a compound of Formula 2 or 2′, as below, or a mixture thereof:
HO[CH₂—CH₂O]ₘ[CH(CH₃)—CH₂O]ₙ[CH₂CH₂O]_{m′}H Formula 1
HO[CH(CH₃)—CH₂O]ₙ[CH₂CH₂O]ₘ[CH(CH₃)—CH₂O]_{n′}H Formula 1′
wherein m and m′ independently represent an integer from about 50 to about 125, e.g. from about 60 to about 115, and n independently represents an integer from 10 to about 80, e.g. from about 20 to about 75;
or
HO[CH₂—CH₂—O]ᵣ—{[CHCH₃—CO—O]_{q}—[CH₂—CO—O]ₚ}—[CH₂—CH₂O]_{r′}H Formula 2
HO[CO—CH(CH₃)—O]_{q}—[CO—CH₂—O]ₚ—[CH₂—CH₂—O]ᵣ—[CO—CH(CH₃)—O]_{q′}—[CO—CH₂—O]_{p′}—H Formula 2′
wherein p and p′ independently represent an integer from about 5 to about 50, q and q′ independently represent an integer from about 5 to about 50, and r and r′ independently represent an integer from about 10 to about 125.

7. A composition as claimed in claim 6, wherein the amphiphilic block copolymer is a compound of Formula 1.

8. A composition according to claim 1, wherein the at least one therapeutic and/or cosmetic agent is selected from the group consisting of analgesics, antimicrobial agents, hormones, enzymes, antiallergic agents, antibiotics, steroidal anti-inflammatory agents, non-steroidal anti-inflammatory agents, cytokines, growth factors, antineoplastic agents, anaesthetics, astringents, immunosuppressants, anticoagulants, anticoagulation factors, clotting factors, anti-oxidants, anti-aging agents and mixtures thereof.

9. A composition according to claim 8, wherein the therapeutic and/or cosmetic agent comprises at least one anti-aging agent selected from the group consisting of retinol, retinal, retinyl palmitate, retinyl acetate, other esters of retinol, retinoic acid, other retinoids, collagen, vitamin E, vitamin C, other vitamins, idebenone, peptides, pentapeptides, polypeptides, transcription factors, UV blockers and mixtures thereof.

10. A composition as claimed in claim 9, wherein the therapeutic and/or cosmetic agent comprises retinol

11. A composition according to claim 8, wherein said therapeutic and/or cosmetic agent comprises at least one non-steroidal anti-inflammatory agent.

12. A composition according to claim 11, wherein said non-steroidal anti-inflammatory agent comprises ibuprofen or a pharmaceutically acceptable salt thereof.

13. A composition according to claim 1, wherein the therapeutic and/or cosmetic agent comprises at least one anaesthetic.

14. A composition according to claim 13, wherein said anaesthetic comprises lidocaine or a pharmaceutically acceptable salt thereof.

15. A composition according to claim 1, wherein the total amount of i) and ii) in said composition forms a polymeric hydrogel at body temperature and wherein the total amount of iii), iv) and v) in said composition does not prevent i) and ii) forming a polymeric hydrogel at body temperature.

16. A composition according to claim 15, wherein said composition is thermoresponsive, having a viscosity at 5° C. of no more than 1000 mPa·s and at 37.5° C. of no less than 10,000 mPa·s.

17. A dermal filler comprising a composition as claimed in claim 1.

18. A process for preparing a composition according to claim 1, comprising mixing i), ii), iv) and, if any, v) to form an aqueous solution, suspension or dispersion, and mixing and dissolving iii) in said aqueous solution, suspension or dispersion.

19. A process for preparing a composition according to claim 18, comprising dissolving ii) in i) to form a resultant solution thereof, said dissolving being effected at a temperature wherein the viscosity of the resultant solution is less than 1000 mPa·s.

20. A process as claimed in claim 19, wherein iii), iv) and, if any, v) are added to the resultant solution to form a final solution, suspension or dispersion, said addition being effected at a temperature wherein the viscosity of the final solution, suspension or dispersion is less than 1000 mPa·s.

21. A process as claimed in claim 19, wherein at least one of iii), iv) and, if any, v) is/are dissolved, suspended and/or dispersed in i) prior to dissolving ii) in i) to form a final solution, suspension or dispersion, said dissolving being effected at a temperature wherein the viscosity of the final solution, suspension or dispersion is less than 1000 mPa·s.

22. A method of treating a patient suffering from or potentially suffering from internal injury or infection comprising administering a composition as claimed in claim 1 into the body of the patient substantially at the point of injury or infection or near there to.

23. A method as claimed in claim 20, wherein the composition is administered via intra-articular injection.

24. A method of reducing wrinkles or the appearance of wrinkles on a facial or other area of skin of a patient, comprising injecting a composition as claimed in claim 15 into the skin of the patient below the surface of the skin at a point below or near to the wrinkle.

25. A composition as claimed in claim 9, wherein the therapeutic and/or cosmetic agent comprises retinyl palmitate.

26. A method for reducing wrinkles or the appearance of wrinkles on a facial or other area of skin of a patient, comprising the topical application of a composition as claimed in claim 15 at or near to the wrinkle.

27. A method for treating a patient suffering from or potentially suffering from at least one aliment of the eye comprising the topical application of a composition according to claim 15 into the interior or onto the surface of the eye.
